(19) United States
(12) Patent Application Publication
(10) Pub. No.: US 2011/0306933 A1
(43) Pub. Date: Dec. 15, 2011

(54) Safety cannula with automatic retractable needle

(76) Inventors: Ilija Djordjevic, East Granby, CT (US); Sushil K. Kanwar, West Hartford, CT (US)

(21) Appl. No.: 12/802,676

(22) Filed: Jun. 11, 2010

(51) Int. Cl. A61B 17/34

(52) U.S. Cl. 604/164.08

(57) ABSTRACT

An intravenous cannula device (10) having a cannula body (12), a needle carrier (20), and a needle carrier container (36) nested in series. The needle carrier is spring loaded (48) toward the container, and the container carries an external lever assembly (50) that triggers the spring as the cannula is pushed forward of the container toward full insertion in the patient, whereby the released spring drives the needle carrier including needle (30) into the container.

### BACKGROUND

The present invention relates to intra venous (IV) cannulas that incorporate a safety feature whereby the needle is retracted after the cannula has been fully inserted into the patient.

A typical IV cannula features a steel needle with a sharp tip protruding through a slightly larger diameter flexible cannula tube. After the skin has been penetrated and the flexible tube inserted, the needle can be removed and discarded. There are however two major areas of concern. First there is a danger of injury because of the exposed sharp tip and secondly there is also a risk of infection because the outside diameter of the needle which was in contact with patient's blood might be contaminated. Safety cannulas are known in which if the needle was removed from the patient following proper procedure, the needle tip will remain within a special cavity in the needle carrier, where a protective flap pivots into position. Such safety cannulas are somewhat complex, but more importantly, if the proper procedure is not carefully followed, it is also possible to remove the needle without triggering the automatic cover flap because the separation between the cannula housing and the needle carrier can occur in more than one place.

Yet another disadvantage with the above described cannula arises from the needle diameter being deformed in one location to prevent the metallic needle tip protection shield from slipping off the needle and exposing the sharp tip. This deformation however obstructs the flow through the needle so blood has to flow through the gap between needle and the flexible tube and because of that it can take longer before the blood flows into the cannula body chamber, thereby confirming that the target was indeed been hit and the needle can now be removed.

### SUMMARY

The present invention effectively overcomes these functional disadvantages while substantially reducing the number of components required for assembly.

In a general aspect, the invention is directed to an intravenous cannula device having a cannula body, a needle carrier, and a needle carrier container nested in series, wherein the needle carrier is spring loaded toward the container, and the container carries an external lever assembly that triggers the spring as the cannula is pushed forward of the container toward full insertion in the patient, whereby the released spring drives the needle carrier including needle into the container

The disclosed device has a cannula body with a forward projecting tube, a needle carrier that is spring loaded toward a needle container having a receiving compartment that extends behind the needle carrier, and a lever assembly having locking arms that extend longitudinally along the exterior of the needle container. Each arm has a base that is longitudinally fixed with respect to the container, a free end having a hook that passes through openings in the container and engages a shoulder on the needle carrier to hold the needle carrier and thus prevent entry into the compartment of the container, and a trigger for releasing the spring, actuated by longitudinal motion of the cannula body relative to the container.

Preferably, the container, needle carrier, and lever assembly are longitudinally slidable together relative to the cannula body such that beyond a predetermined position of the container relative to the cannula body the trigger is actuated and thereby releases the hooks from the shoulder, whereby the spring drives the needle carrier including needle into the cavity of the container.

The implementing embodiments of a safety cannula device as disclosed herein, include a hollow cannula body having an elongated tube extending forward from the body. A tubular needle carrier is axially aligned behind cannula body, and supports a needle that extends through the cannula body and cannula tube to a free end that projects forwardly of the cannula tube. A tubular needle container is fixed to and encapsulates the needle carrier and has a front portion slidingly engaging the cannula body, an intermediate portion defining a cavity, and a back portion having a length at least equal to the extension of the needle through the cannula tube. In the cavity, the needle carrier has a shoulder and a spring acts between the container and a shoulder on the needle carrier, biasing the carrier toward the back portion of the container. The arms of the lever assembly extend longitudinally along the exterior at the intermediate portion of the needle container, each arm having a base that is longitudinally fixed with respect to the container, a free end having a hook that passes through one of said openings and engages the shoulder.

The cannula device can be held by a nurse between the thumb and middle finger at the locking arms. After blood is drawn through the needle and appears in a view window, the nurse gradually pushes the cannula off the needle. The container, needle carrier, and lever assembly together slide relative to the cannula body such that beyond a predetermined position of the cannula relative to the container the trigger is actuated and thereby releases the hooks from the shoulder, whereby the spring drives the needle carrier including needle into the back portion of the container.

The advantages include:
Passive safety feature (automatic needle retraction)
Lost motion provides partial needle tip retraction and by that provides support for smaller diameter cannula tubes
Complete encapsulation of potentially contaminated needle after use
Unchanged cannula (catheter) body (identical with current production cannulas)
Identical components can be used with either wingless or wing type cannulas
Modular design option minimizes tooling costs
Aesthetically pleasing, compact design

### BRIEF DESCRIPTION OF THE DRAWING

Embodiments will be described with reference to the accompanying drawing, in which:

FIG. 1 is an oblique view of a first embodiment of a complete cannula device as removed from a sterile pouch, ready for use;

FIG. 2 is an oblique view similar to FIG. 1, in longitudinal section;

FIG. 3 is a longitudinal section view of the region of the device where the cannula body, needle carrier, carrier container and lever assembly are interconnected in the condition shown in FIG. 2;

FIG. 4 is an oblique schematic view showing how the locking arms of the lever assembly are prevented from pivoting radially outward so long as the trigger arms are in contact with the canula body in the condition shown in FIG. 2;

FIG. 5 is an illustration of how the medical technician can hold the device in the condition shown in FIG. 2, immediately before insertion of the needle into the patient;

FIG. 6 is a longitudinal section view showing a condition after the technician has pushed the cannula body forward relative to the container, needle carrier, and lever assembly, immediately before triggering of the spring release of the lever assembly;

FIG. 7 shows the safe condition following the exit of the cannula body from the container, in which the lever assembly has been triggered and the spring has driven the needle carrier with needle fully into the container;

FIG. 8 is an oblique view of the preferred needle carrier;

FIG. 9 shows the details of the type of lever assembly of the first embodiment, with an alternative shape for the hooks;

FIGS. 10-12 show a second embodiment for the lever assembly;

FIG. 13 shows a third embodiment of the lever assembly; and

FIG. 14 shows a variation of the container, having a variable length snap extension to accommodate different length needles.

### DETAILED DESCRIPTION

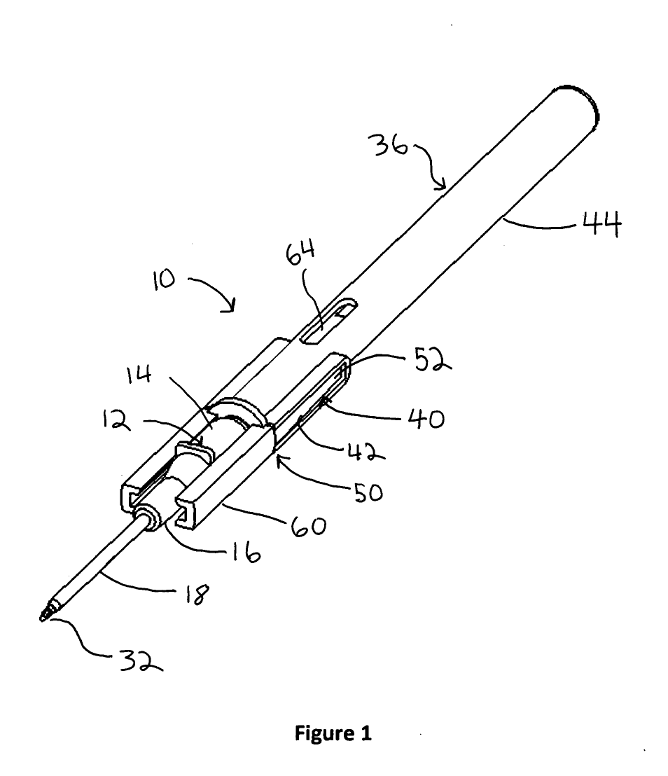

FIGS. 1-8 show a first embodiment of a safety IV cannula according to the present disclosure. The safety cannula device 10 comprises a hollow cannula body 12 having a larger diameter back portion 14, a smaller diameter front portion 16, and an elongated tube 18 extending forward from the front portion of the body. A tubular needle carrier 20 having front 22, intermediate 24, and back portions 26, is nested on the body, with the front portion 22 within the back portion 14 of the cannula body and the intermediate portion 24 longitudinally spaced in cavity 28 behind the back portion 14 of the cannula body. The front portion 22 supports a needle 30 that extends through the cannula body 12 and cannula tube 18 to a free end 32 that projects forwardly of the cannula tube 18. The intermediate portion 24 forms a shoulder 34 in cavity 28.

A tubular needle container 36 is nested with the cannula body 12 and the needle carrier 20, having a front portion 38 captured between the front portion 22 of the needle carrier and the back portion 14 of the cannula body, an intermediate portion 40 having a plurality of elongated external channels 42, and a back portion 44 defining a compartment having a length at least equal to the extension of the needle 30 through the cannula tube 18. The channels 42 each have a radial opening 46 into the cavity 28, adjacent to the shoulder 34 on the needle carrier. A helical or coil spring 48 has one end seated near the front end 38 of the carrier at the forward end of the cavity 28 that extends through the intermediate portion 40 of the container. The other end of the spring 48 acts axially on the needle carrier 20, biasing the carrier toward the back portion 44 of the container.

A lever assembly 50 has locking arms 52a, 52b that extend longitudinally in respective channels 42 of the needle container 36, each arm having a base 54 that is longitudinally fixed with respect to the container 36, and a free end 56 having a hook 58 that passes through one of the openings 46 and engages the shoulder 34 of the needle carrier. The shoulder 34 imposes a radially outward force component on each hook 58 while a longitudinally directed force component resists the bias of the spring and prevents the needle carrier 20 from entering the back portion 44 of the container 36, until the locking arms are triggered. The trigger 60 is actuated passively and automatically by longitudinal motion of the cannula body 12 relative to the container 36, as the medical technician advances the cannula into the patient. This is possible because the container 36, needle carrier 20, and lever assembly 50 together are longitudinally slidable relative to the cannula body 12 such that beyond a predetermined position P of the cannula body 12 relative to the container (or trigger) the trigger 60 is actuated and thereby adds another radially outward force component to the hooks 58 such that the hooks move radially outward from the shoulder 34. This releases the intermediate portion 24 of the needle carrier, whereby the spring 48 drives the needle carrier 20 including needle 30 into the back portion 44 of the container. Preferably, the trigger comprises a plurality of trigger arms 60a, 60b in alignment with the corresponding plurality of locking arms 50a. 50b, with the trigger arms extending forward beyond the front portion 38 of the container.

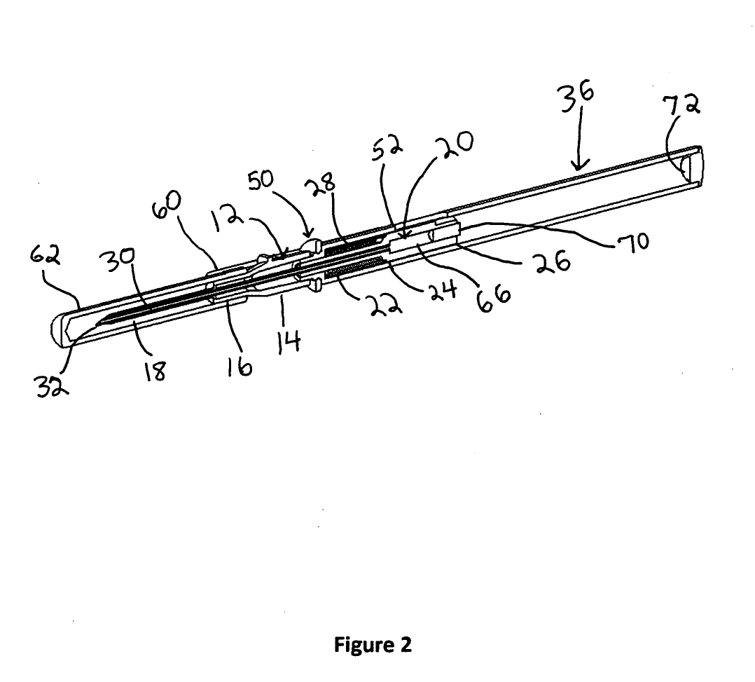

FIG. 1 shows that the sharp needle tip 32 initially projects from the cannula tube 18. The device is, however, initially distributed in sterile packaging with a tubular safety cover 62 as shown in FIG. 2. The cover 62 is held by friction between the front portion 16 of the cannula body and the front portion of the trigger arms 60 that overhang the front portion 38 container. This cover must be removed and discarded before the use.

In use, the device 10 automatically retracts the needle carrier 20 into the container 36. The locking arms 52 and trigger arms 60 are connected via a circular ring 76. The inner circumference 76 defines the base 54 of each locking arm, and is captured in a groove 78 on the exterior of the front portion 38 of the container 36, thus fixing the lever assembly 50 with respect to the container 36. The central ring 76 of the integral lever pair is snapped into a corresponding circular groove 78. As a result, after the needle container 36 is removed from the cannula body 12 the lever assembly 50 will remain attached to the container, avoiding loose parts.

A view window 64 is provided on the container 36, where the transparent back portion 26 of the needle carrier forms an axially extending bore or chamber 66. A porous plug 70 is located at the back end of the chamber and protrudes about 1 to 2 mm beyond the back edge of the carrier. This plug 70 has a dual function. The porosity of the plug allows air displaced by the blood flowing into the view chamber 66 after the sharp tip 32 has penetrated a patient's vein to escape and it also dampens the impact against the back wall 72 of container after the spring 48 has been released.

The cannula device can be held by a nurse between the thumb and middle finger at the container alongside the locking arms 52. After blood is drawn through the needle 30 and appears in the view window 64, the nurse gradually pushes the cannula 12 off the needle carrier 36, preferably by pushing with the index finger on external tab 74 formed on the cannula body 12, and accessible between the trigger arms 60 of the lever assembly. The container 36, needle carrier 20, and lever assembly 50 together slide relative to the cannula body 12 such that beyond a predetermined position P the trigger 60 is actuated and thereby releases the hooks 58 from the shoulder 34, whereby the spring 48 drives the needle carrier 20 including needle 30 into the back portion 44 of the container. Preferably, the trigger actuates when the back end of the cannula body passes the front ends of the trigger arms 60.

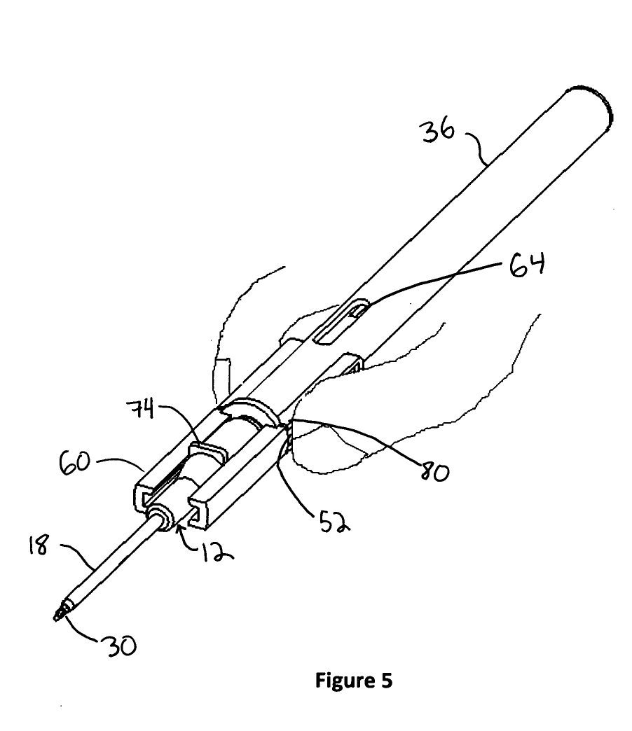

The channel walls 80 are radially outside the neutral position of the locking arms 52. Because the walls act as barriers, the locking arms are able to open up and pull the hooks 58 off the shoulder 34, even if the nurse continues to hold the device as shown in FIG. 5. If the barriers 80 were the same height or lower than the locking arms 52 then the nurse would squeeze the levers directly which could prevent or delay unhooking at the shoulder 34.

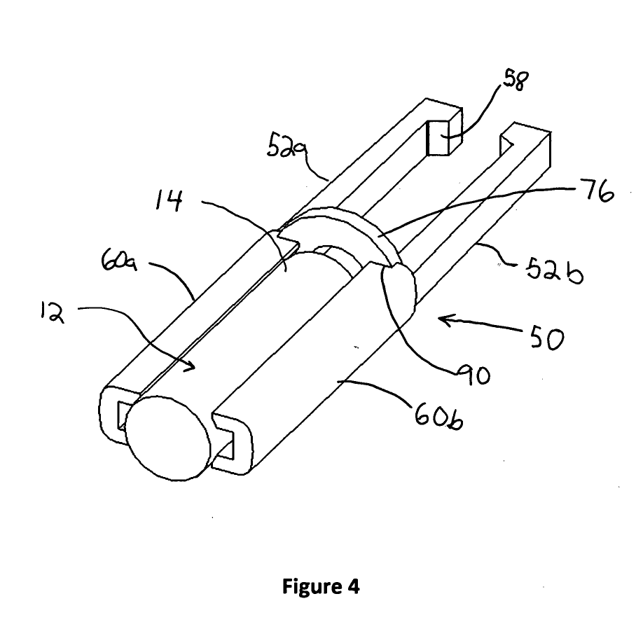

As shown in FIG. 4, each locking arm 52 and its aligned trigger arm 60 is connected through ring 76 to form one long lever that pivots at the ring. The radial outward acting force generated by the spring tries to spread the locking levers 52. This is however not possible so long the cannula body 12 (represented here as a cylinder) prevents the trigger arms 60 from moving closer together. Once the cylinder is removed the locking arms open up and release the spring. Until this occurs, the radial force is trying to stretch the connecting ring 76 but because this radial force is very small (about 0.04 N or 1.4 oz) and the rigidity of the connecting ring in this direction is adequate no deformation takes place. The rigidity of the ring in the arm bending direction is however small and the ring will deform easily.

By characterizing the lever assembly 50 in this embodiment as integral, the inventors mean that the locking arms 52a, 52b, respective trigger arms 60a, 60b and ring 76 are either formed as a unitary part or formed of distinct components that are substantially permanently joined. In this context, permanently joined means the lever assembly 50 is self-contained in normal use, i.e., the components are not expected to come apart.

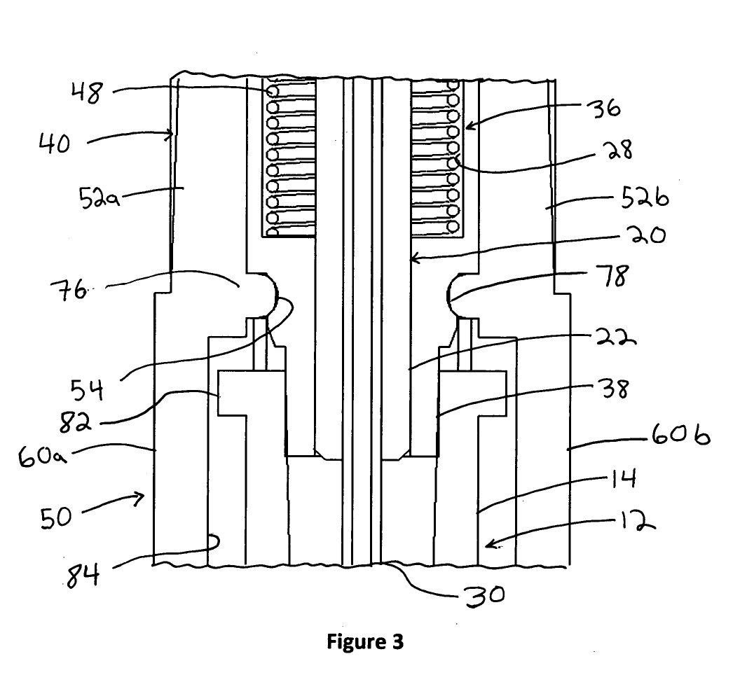

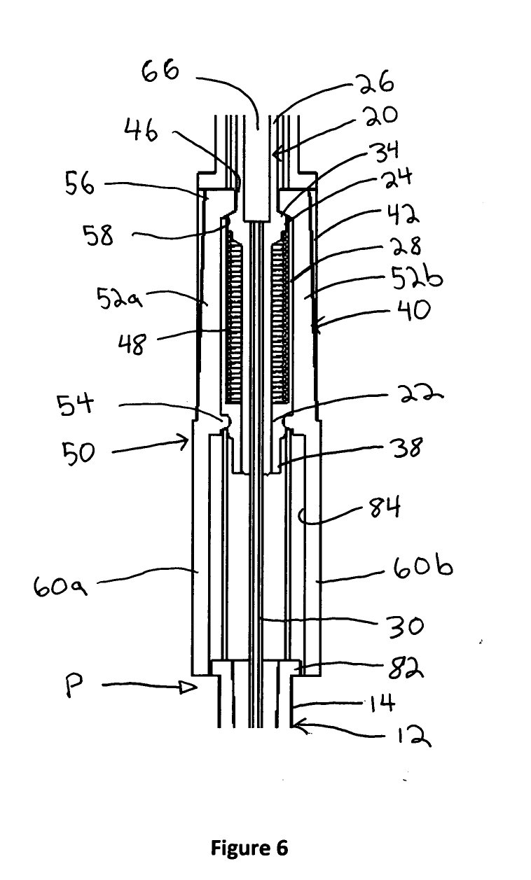

FIG. 3 shows the initial relationship of the cannula body 12 to the lever assembly 50, container 36, and needle carrier 20 and FIG. 6 shows this relationship at the moment just before the back portion 14 of the cannula 12 exits the lever assembly 50 to thereby release the spring. The cannula 12 has external ears 82 that ride in internal grooves 84 of the trigger arms. The ears need not bear radially against the grooves. The reason for the ears 82 riding in and being supported by the grooves 84 is to prevent premature spring release. When the needle container 36 is moved back by the thumb and the middle finger as shown in FIG. 5 the index finger (not shown) is holding the cannula body 12 by the protrusion 74. Especially with thin needles and in the absence of these grooves 84, it is possible that the force applied by the index finger inadvertently deflects the needle and by that laterally pushes the cannula body out of engagement with, and thereby prematurely triggering, the trigger arms 60.

Because of the inclination of the surfaces on both parts 58, 34 the spring force creates a radial force component trying to spread the levers which however is prevented so long the cylindrical portion of cannula body 12 is captured by the trigger arms 60 of the lever assembly 50.

Once the ears 82 completely exit the guide grooves 84, the radial force component at the shoulder 34 arising from the bias of the spring 48 will spread the locking arms 52 and release the spring. Any potential opposite radial force appearing between the hooks 58 due to deformation of the connecting ring 76 after the spring is released should be minimized to insure full retraction of the needle. The force must not be able to stop the motion by interference with the spring coils.

As the spring is never in contact with blood a lesser grade of stainless steel can be used instead of otherwise required medical grade stainless steel.

The lost motion of the cannula 12 until spring release allows for full insertion of the cannula tube 18 in the vein because the tube is partially supported by the more rigid needle 30. This feature is especially important for the smaller diameter cannula tubes which lack the rigidity to support insertion forces and because of that could collapse before the tube is fully inserted.

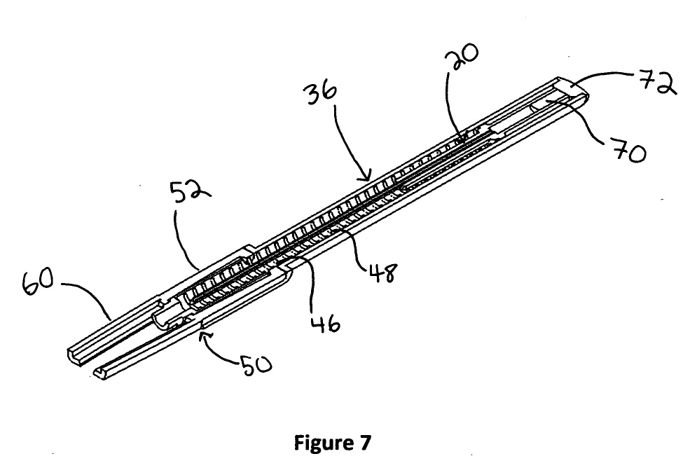

FIG. 7 shows the condition of the container 36, needle carrier 20, and lever assembly 50 after the cannula 12 was removed and the spring 48 was released. This shows the ideal natural (neutral) shape of the components of the lever assembly 50, i.e., the trigger arms 60 are inclined toward the axis while the locking arms 52 are contained substantially entirely within the openings 46 in the carrier 36.

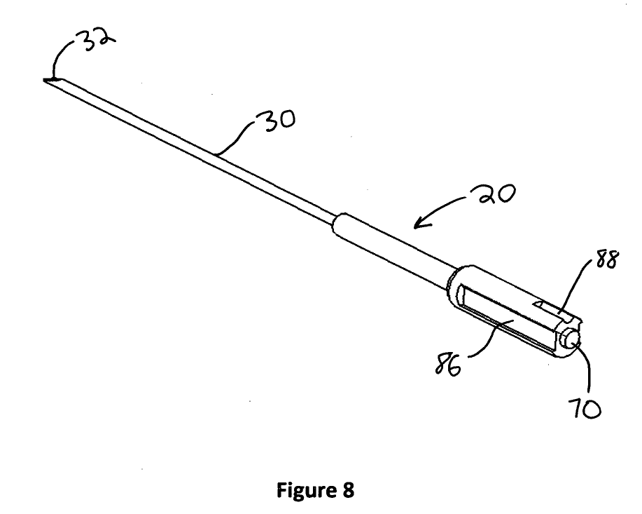

As shown in FIG. 8, the needle carrier 20 can have two lateral grooves 86 or similar recesses, which are situated in back of the shoulder 34 of the assemble device and accommodate the hooks 58 of both locking levers 52 to keep the spring compressed. A visual or structural marker, such as notch 88, can also be provided to insure proper orientation of the obliquely cut needle tip 32 during assembly. The locking arm channels 42 of the needle carrier are spaced 180° apart and because of that it would be possible to install the needle carrier 20 with the needle tip 32 wrongly pointing up. In order to prevent this mistake, the notch 88 on the needle carrier is used as a signal indicating the correct position of the sharp point. This reference notch or groove can engage with a corresponding protrusion of the assembly tool to insure the proper orientation.

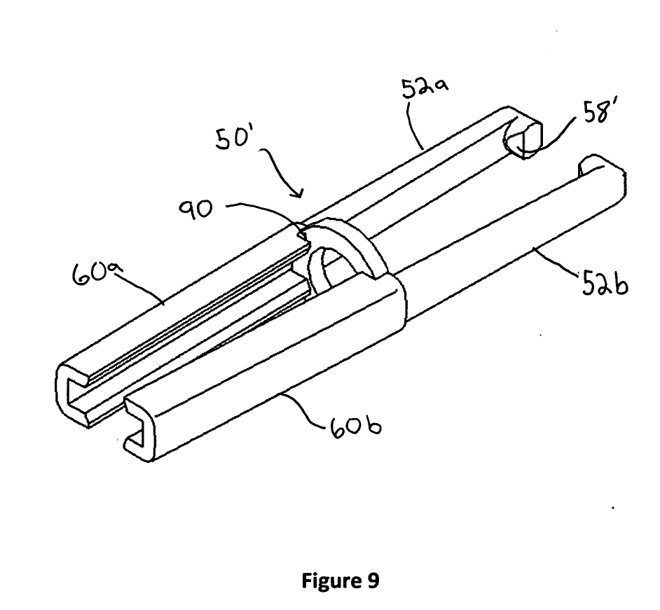

FIG. 9 shows the details of the type of lever assembly of the first embodiment, with an alternative shape for the hooks. In this lever assembly 50′, there are only two differences relative to FIG. 4. The hooks 58 of FIG. 4 have a parabolic surface, whereas the hooks of FIG. 9 have a rectangular surface. FIG. 9 also shows in greater detail how the lever assemblies 50 and 50′ are composed of three parts, i.e., one lever consisting of arms 52a and 60a; another lever consisting of arms 52b and 60b; and a central ring 76 snap fit into notches or grooves 90 in arms 60 at the transition to arms 52.

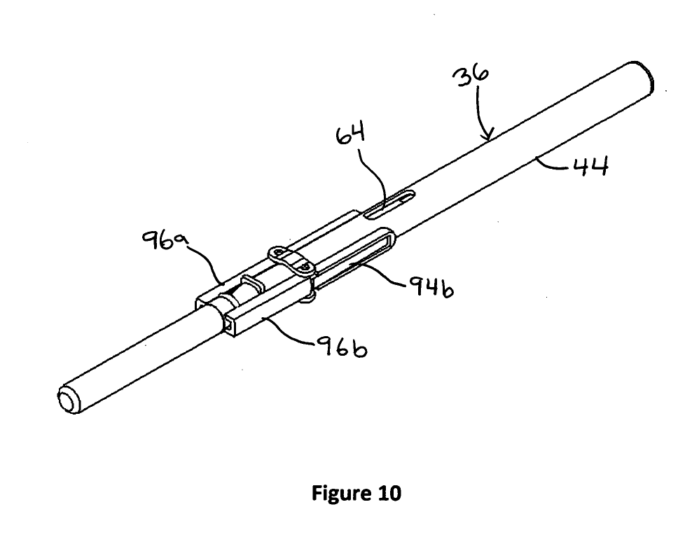

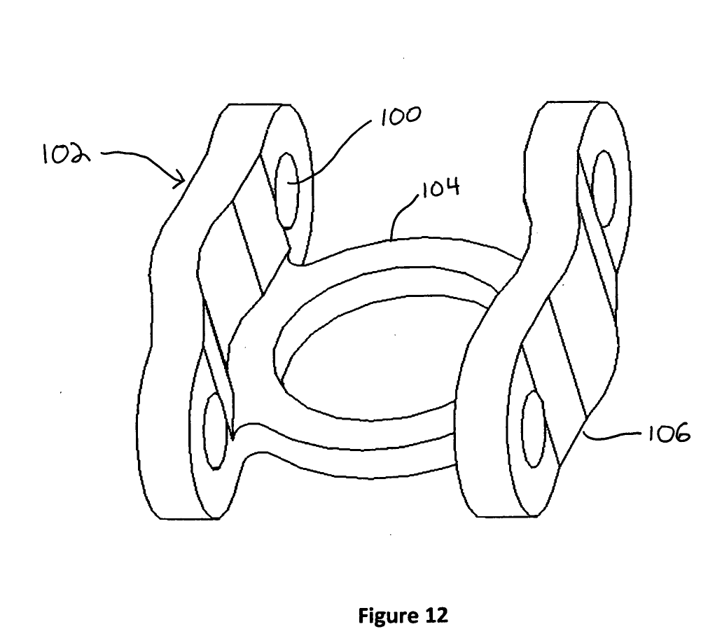

FIGS. 10-12 show another embodiment 92 of the lever assembly, consisting of one lever having integral locking and trigger arms 94a, 96a and another lever having integral locking and trigger arms 94b, 96b. At the transition between the arms of each lever, a pair of opposed posts 98 project laterally. The posts 98 engage respective pairs holes 100 in cage 102. The cage 102 has a central ring 104 that is coaxial with the longitudinal axis of the device, and two wings 106 that extend tangentially to the ring and transverse to the axis of the device. Each wing has a hole at opposite ends, whereby four posts 98 engage four holes 100. In the assembled condition, the wings and posts define a four sided frame that firmly surrounds container 20. Each pair of posts can pivot at the respective pair of holes. The advantage of this embodiment is the absence of any radial force which could possibly prevent complete retraction of the needle as a consequence of the locking hooks interfering with the coils of the spring.

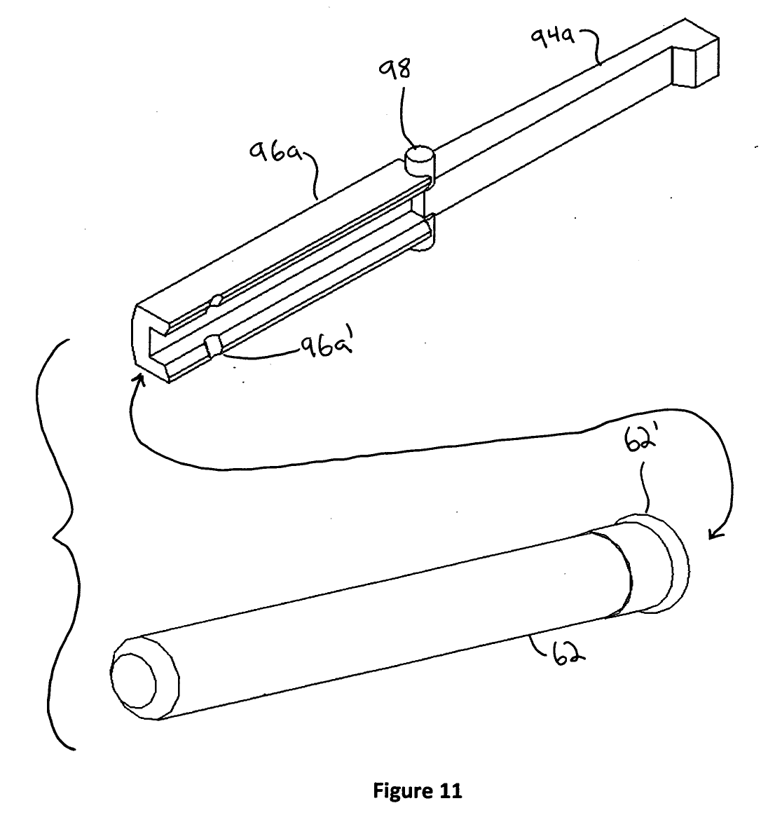

FIG. 11 also shows that the front end 96a′ of each the trigger arm can have a detent type cooperation with the back end 62′ of the safety cover, to substitute for or enhance the previously described friction fit.

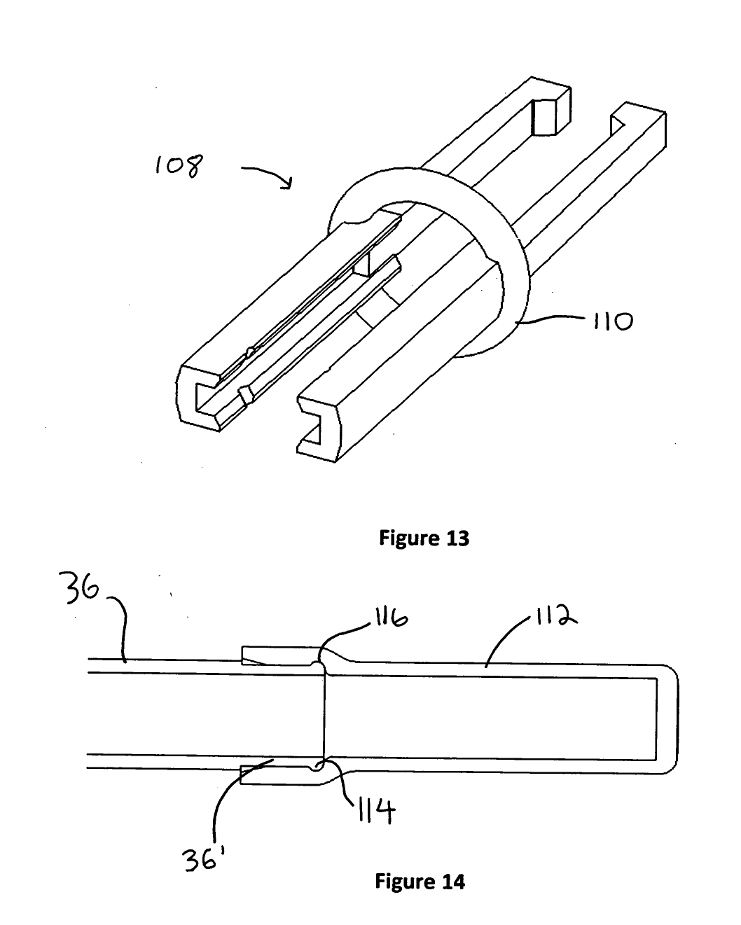

FIG. 13 shows a third embodiment 108 of a lever assembly, wherein the cage of FIG. 11 has been reduced to the form of a simple elastic ring 110 at the transition (pivot point) of the lever arms. The ring 110 provides a radially inward force at its entire inner surface, thereby securing the lever assembly 108 longitudinally on the container but permitting the necessary pivoting action to release the spring. This embodiment has the advantage of inexpensive tooling, however the components will likely fall apart after the cannula has been removed from the patient.

FIG. 14 shows an alternative embodiment of the container, in which an extension tube 112 is snap fit on the open back end 36′ of the container 36. Preferably, the back end 36′ has a reduced OD that easily fits into the ID of a complementary bore ID at the front of the tube. Cooperating positive 114 (protruding) and negative 116 (receding) structure care engaged as the tube is slid onto the back end 36′ of the container 36. Any form of detent type cooperation is suitable for this purpose. This embodiment provides a very simple variable length snap on extension of the length of the container 36. As a consequence, the tooling for the fabrication and assembly of the lever assembly and associated triggering components can be identical for cannula devices having different lengths of needles.

1. An intravenous cannula device comprising:
a cannula body having a frontally projecting tube for insertion in a patient;
a needle carrier axially aligned behind the cannula body and having a needle in and projecting from the cannula tube;
a needle container in coaxial, nested relation to the cannula body and the needle carrier;
wherein the needle carrier is spring loaded backward away from the cannula, and the container carries an external lever assembly that triggers the spring as the cannula body is pushed forward of the container toward full insertion in the patient, whereby the triggered spring drives the needle carrier including needle fully into a compartment of the container.

2. The cannula device of claim 1, wherein the lever assembly has locking arms that extend longitudinally in respective channels of the needle container, each locking arm having
a base that is longitudinally fixed with respect to the container;
a free end having a hook that passes through an opening in the container and engages a shoulder on the needle carrier, which hook resists the load of the spring and prevents the needle carrier from entering the compartment of the container; and
a trigger actuated by longitudinal motion of the cannula body relative to the container.

3. The cannula device of claim 2, wherein said container, needle carrier, and lever assembly together are longitudinally slidable relative to said cannula body such that beyond a predetermined position of the cannula body relative to the container the trigger is actuated and thereby imparts a radially outward force component to the hooks such that the hooks move radially outward from the shoulder and release the needle carrier, whereby the spring drives the needle carrier including needle into the compartment of the container.

4. The cannula device of claim 2 wherein the lever assembly consists essentially of:
one lever defining a locking arm and integral, longitudinally aligned trigger arm;
another lever defining another locking arm and integral, longitudinally aligned trigger arm;
a central ring having an outer portion that fixedly engages the levers and an inner portion defining said base that fixedly engages the container and thereby longitudinally fixes the lever assembly to the container.

5. The cannula device of claim 2, wherein the lever assembly consists essentially of:
one integral lever having a locking arm that transitions into a trigger arm;
another integral lever having a locking arm that transitions into a trigger arm;
each lever having a pair of posts projecting laterally at the respective transitions and defining said base;
a cage having a central ring that is coaxial with the longitudinal axis of the device, and two wings that extend tangentially to the ring and transverse to the axis of the device, each wing having a pair of holes;
whereby the pairs of posts engage the pairs of holes such that in the assembled condition, the wings and posts define a four sided frame that firmly surrounds and thereby longitudinally fixes the lever assembly to the container.

6. The cannula device of claim 2, wherein the lever assembly consists essentially of:
one integral lever having a locking arm that transitions into a trigger arm;
another integral lever having a locking arm that transitions into a trigger arm;
an elastic ring surrounding and providing a radially inward force on the levers at the transitions, which force is transmitted to the container through the transitions such that the transitions define said base and secure the lever assembly longitudinally on the container.

7. The cannula device of claim 2, wherein the container comprises a body having an open back end and a variable length extension including an extension tube having an open front end in overlapping engagement with the open back end of the container body and a closed back end.

8. The cannula device of claim 7, wherein
the back end of the container body has a reduced OD and the front portion of the extension tube has a complementary ID that fits on the back end of the container; and
the back end of the container body and the front portion of the extension tube have cooperating positive and negative detent structures that are engaged as the extension tube is slid onto the back end of the container.

9. A safety cannula device comprising:
a hollow cannula body having an elongated tube extending forward from the body;
a tubular needle carrier axially aligned behind cannula body, and having front and back portions, wherein the needle carrier supports a needle that extends through the cannula body and cannula tube to a free end that projects forwardly of the cannula tube, and wherein the needle carrier has a shoulder;
a tubular needle container fixed to and encapsulating the needle carrier and having a front portion slidingly engaging the cannula body, an intermediate portion defining a cavity in which said shoulder is located, and a back portion defining a container having a length at least equal to the extension of the needle through the cannula tube, and at least one radial opening in the intermediate portion, laterally of the shoulder on the needle carrier;
a spring situated within the cavity of the container and acting between the container and the needle carrier, biasing the carrier toward the back portion of the container;
a lever assembly having locking arms that extend longitudinally along the exterior at the intermediate portion of the needle container, each arm having a base that is longitudinally fixed with respect to the container, a free end having a hook that passes through one of said openings and engages the shoulder of the needle carrier to prevent the needle carrier from entering the back portion of the container, and a trigger actuated by longitudinal motion of the cannula body relative to the container.

10. A safety cannula device comprising:
a hollow cannula body having an elongated tube extending forward from the body;
a tubular needle carrier axially aligned behind cannula body, and having front, intermediate, and back portions, wherein the front portion supports a needle that extends through the cannula body and cannula tube to a free end that projects forwardly of the cannula tube, and wherein the intermediate portion has a shoulder;
a tubular needle container encapsulating the needle carrier and engaging the cannula body, having a front portion, an intermediate portion having a plurality of elongated external channels, and a back portion defining a compartment having a length at least equal to extension of the needle through the cannula tube, wherein the channels each have a radial opening laterally of the shoulder on the needle carrier;
a spring situated within the intermediate portion of the container and acting between the front portion of the container and the needle carrier, biasing the carrier toward the back portion of the container;
a lever assembly having locking arms that extend longitudinally in respective channels of the needle container, each arm having a base that is longitudinally fixed with respect to the container, a free end having a hook that passes through one of said openings and engages the shoulder of the needle carrier to prevent the needle carrier from entering the back portion of the container, and a trigger actuated by longitudinal motion of the cannula body relative to the container;
said container, needle carrier, and lever assembly together being longitudinally slidable relative to said cannula body such that beyond a predetermined position of the cannula body relative to the container the trigger is actuated and thereby releases the hooks from the shoulder, whereby the spring drives the needle carrier including needle into the compartment of the container.

11. The safety cannula device of claim 10, wherein the back portion of the needle carrier includes two external grooves that are aligned with the channels of the container.

12. The safety cannula of claim 10, wherein a porous plug projects axially from the back portion of the needle carrier.

13. The safety cannula of claim 11, wherein a porous plug projects axially from the back portion of the needle carrier.

14. The safety cannula of claim 10, wherein the back portion of the needle carrier includes a needle alignment mark.

15. The safety cannula of claim 10, wherein the trigger includes two longitudinally extending trigger arms that contact and are slidable along the cannula body and that actuate a pivot action on the locking arms when the cannula body slides out of contact with the trigger arms at said position.

16. The safety cannula of claim of 15, including a tubular cover having a front end that extends forward of the needle, and a back end that overlaps the front portion of the cannula body and is overlapped by front ends of the trigger arms.

17. the safety cannula of claim 15, wherein the front end the trigger arms and the back end of the cover are engaged with a snap connection that is manually broken to remove the cover immediately before use of the cannula device.

18. A safety cannula device comprising:
a hollow cannula body having a larger diameter back portion, a smaller diameter front portion, and an elongated tube extending forward from the front portion of the body;
a tubular needle carrier having front, intermediate, and back portions, with the front portion within the back portion of the cannula body and the intermediate portion in a cavity behind the back portion of the cannula body, wherein the front portion supports a needle that extends through the cannula body and cannula tube to a free end that projects forwardly of the cannula tube, and wherein the intermediate portion forms a shoulder in said cavity;
a tubular needle container having a front portion captured between the front portion of the needle carrier and the back portion of the cannula body, an intermediate portion having a plurality of elongated external channels, and a back portion defining a compartment having a length at least equal to extension of the needle through the cannula tube, wherein the channels each have a radial opening into the cavity adjacent to the shoulder on the needle carrier;
a spring situated within the intermediate portion of the container and acting axially on the needle carrier, biasing the carrier toward the back portion of the container;
a lever assembly having locking arms that extend longitudinally in respective channels of the needle container, each arm having a base that is longitudinally fixed with respect to the container, a free end having a hook that passes through one of said openings and engages the shoulder of the needle carrier, which shoulder imposes a radially outward force component on the hook while a longitudinally directed force component resists the bias of the spring and prevents the needle carrier from entering the back portion of the container, and a trigger actuated by longitudinal motion of the cannula body relative to the container;
said container, needle carrier, and lever assembly together being longitudinally slidable relative to said cannula body such that beyond a predetermined position of the cannula body relative to the container the trigger is actuated and thereby adds another radially outward force component to the hooks such that the hooks move radially outward from the shoulder and release the intermediate portion of the needle carrier, whereby the spring drives the needle carrier including needle into the back portion of the container.
